(19) United States
(12) Patent Application Publication
(10) Pub. No.: US 2016/0192959 A1
(43) Pub. Date: Jul. 7, 2016

(54) Twin Forceps for Single Access Laparoscopy

(71) Applicants: Guido DANIELI, Rende (CS) (IT); CALABRIAN HIGH TECH SRL, Rende (CS) (IT)

(72) Inventors: Guido DANIELI, Rende (CS) (IT); Antonia RIZZUTO, Rende (CS) (IT); Mario DONNICI, Rende (CS) (IT); Paola NUDO, Rende (CS) (IT); Pasquale Francesco GRECO, Rende (CS) (IT); Rosario SACCO, Rende (CS) (IT)

(73) Assignee: CALABRIAN HIGH TECH SRL, Rende (CS) (IT)

(21) Appl. No.: 14/916,192

(22) PCT Filed: Sep. 3, 2014
(86) PCT No.: PCT/IT2014/000230

§ 371 Date: Mar. 3, 2016

(30) Foreign Application Priority Data
Sep. 5, 2013 (IT) CS2013A000023
Aug. 27, 2014 (IT) CS2014A000026

(51) Int. Cl. A61B 17/29

(52) U.S. Cl. CPC A61B 17/29; A61B 2017/00407

(57) ABSTRACT

The patent is relative to a new instrument for single access laparoscopy that tries to overcome the limits of the present instrumentation of this technique, that usually allows to use only one forceps, which does not allow to keep the tissues tenses to work with the surgical instrument.

To overcome this limitation, the proposed instrument presents two arms, presenting a diameter equal to half of the main body, while in the direction perpendicular to this these arms can have an bigger dimension, provided its shape be entirely circumscribed by the external dimension of the initial body, and each bearing at the tip a forceps, mounted on two auxiliary arms. The internal mechanism allows opening the two arms, while the forceps bend toward the inside, keeping their configuration, being all this controlled by a single command. Two additional commands allow then to open and close the forceps individually, and each command can be locked in any position. The forceps can be opened in any direction relative to the plane of the arms, as well as it is possible that one or both of the forceps are replaced by a tool of a different type.

### STATE OF THE ART

The state of the art of SAL (Single Access Laparoscopy) has been already amply illustrated in the patent applications CS2012A000006 of Feb. 22, 2012, CS2012A000032 of Sep. 7, 2012, CS2013A000001 of Jan. 1, 2013, PCT/IT2013/000052 of Feb. 19, 2013, and CS2013A000023 of Sep. 5, 2013 and thus not repeated here.

Purpose of the new patent application is the presentation of the last version of the instrument to make it more reliable and functional its use.

In particular, we are first of all completely separate forceps commands from the one for arms opening, the control of which was entrusted to an internally threaded rotating bezel, whose axial movement is prevented by the outer shell of the handle. The bezel, acting on a worm element provided with special grooves that allow a motion of pure translation with respect to the inner frame of the instrument, on which are hinged the two arms, worm element which, advancing, causes, through connecting rods, the opening of the arms, as it will be better illustrated in the following.

The shape of the command rings was also changed, so that the longest command ring does not interfere with the operator's finger when operating the shorter one. Two ratchets were added to lock the closure of each forceps, providing of course the system with the appropriate unlocking devices. The cables controlling the counter rotation of the arms holding forceps were replaced with appropriate rods suitably shaped. A mechanism that allows to make interchangeable and therefore potentially disposable the forceps was finally introduced, in order to increase the level of security of the system, although designed as a non disposable instrument.

Also you should note how the claimed dependency of this family of patents by the patent family related to US2007299387 A1, is erroneous, since the present individual grippers have nothing to do with that system, because in that case it is a system composed of several elements, of which the central one, also used to obtain the opening of the forceps side, contains the tool operator. In particular we can note that this clamp is instead made solely by the two arms that can be opened without the need of an intermediate member, which in the case of the cited USA patent constrains the position of the instrument operator, placed at the center between the arms, for which if you want to move the zone of action of the instrument operator must also move the position of the side arms. In our case instead arms holder forceps instrument and operator are completely independent, for which the instrument operator can be moved to any point between the two lateral forceps, and even outside of these. Hence the essential novelty of the proposed instrument with respect to the system mentioned above.

### DESCRIPTION OF THE PREFERRED EMBODIMENT

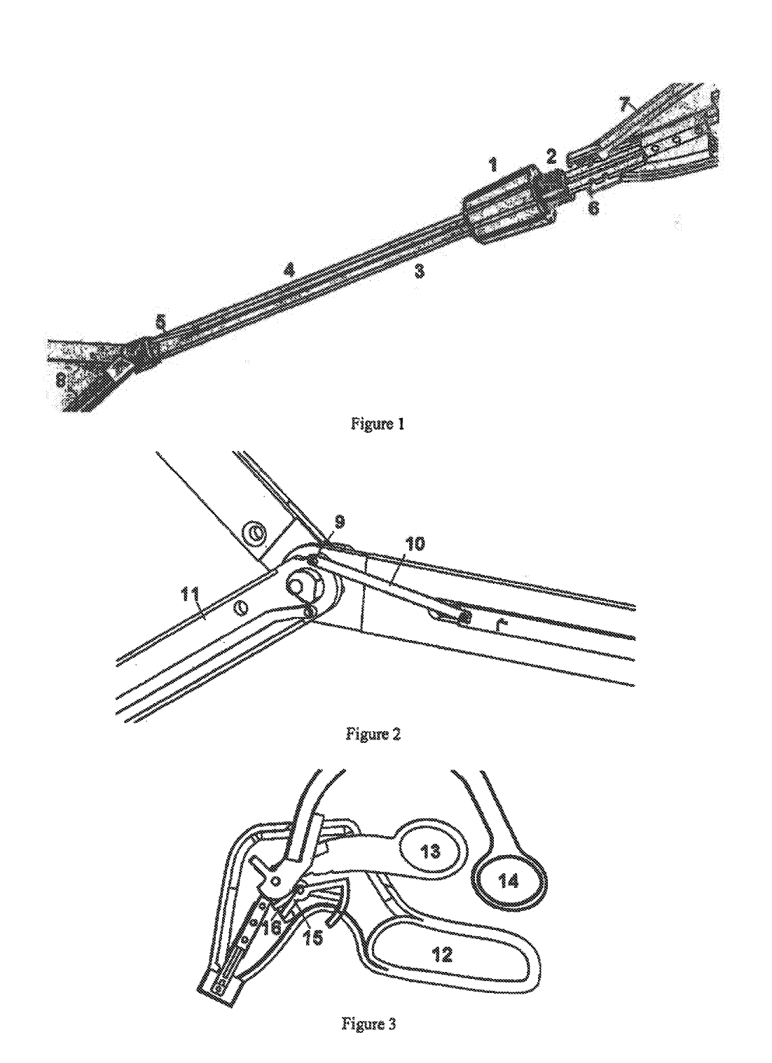

Remaining valid all descriptions of constructive solutions to this tool, contained in patent applications cited above, to which we refer, we observe in FIG. 1 how it is now realized the opening mechanism of the arms, beginning with the description of the system rotating bezel (1)—actuator element (2), which can slide on the inner frame of the gripper (3) being however hindered its rotation. As can be noted, the rotating bezel is internally threaded, while the actuator element presents an outer thread. A rectangular elongated element (4) that ends with a through pin which are hinged the connecting rods of the opening arms (5) is then connected to the actuator element. Conversely offsets of the rotating bezel are prevented, this being fixed, via a collar (6), to the outer cover of the handle (7). Correspondingly, the feed of the connecting rods causes the opening of the arm (8), which are now rotating on coaxial hinges, one for each side of the main frame of the gripper (3), due to the eccentricity of the connecting pin (9) between the connecting rod (10) and arm (11), shown in the following FIG. 2.

FIG. 3 shows the detail of the inner frame to the handle of the instrument (12), which are connected to both rings command closing forceps (13 and 14) that the respective ratchets (15), obviously both equipped with appropriate asymmetric locking teeth, with the springs (16) which implement the ratchet, while the curved elements, part of the same ratchet, allow its the release.

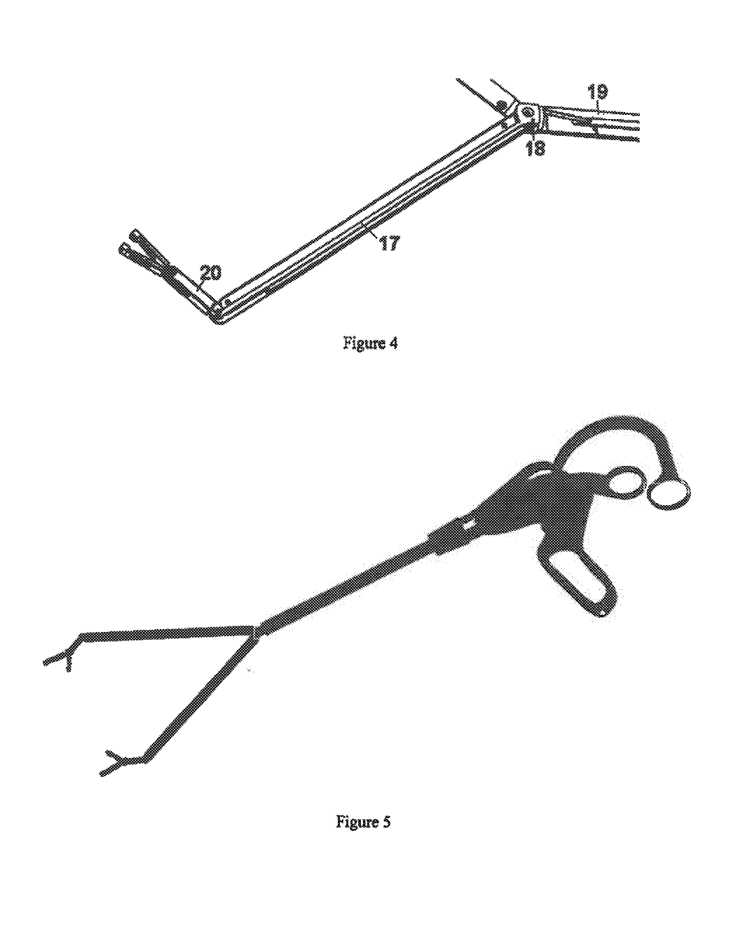

Turning then to FIG. 4, we note how, unlike the mechanism of rotation of the previous frame holder forceps, commanded by cables, connected to the central pin holding the main arms, these cables have been replaced by a connecting rod (17) suitably shaped, that is forced to rotate on a pin fixed to a new closure element of the main body (18), fixed with respect to this (19), being this pin placed at a distance double (3.6 mm) from the axis than that to which is fixed the new connecting rod on the component holder forceps (1.8 mm), and inclined to a radius of −22.5° with respect to the perpendicular to the axis of the main body. When the arm is inclined by −45°, the pin is located at +22.5° with respect to the arm's axis. In the component holder forceps (20) the pin is located initially at −45° in the alignment condition, while it rotates by 90° when the arm is rotated 45° as the ratio between the radii. In this way we obtained a system which is no longer subject to the sliding of the cables as before, so the resulting system could not come back properly aligned after opening of the arms, which is not admissible in a surgical instrument. Being also double the radius in the fixed part with respect to that mobile, you get the rotation of the arm holder forceps opposite and twice that of the main arms. FIG. 5 shows the final aspect of this device.

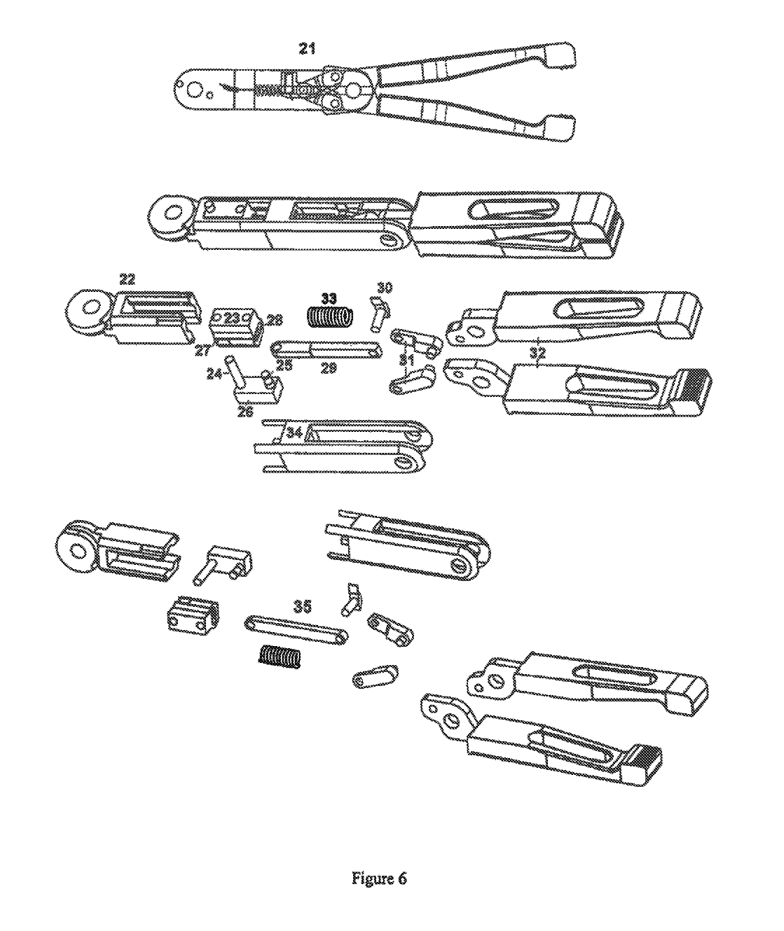

Finally, we note in FIG. 6 as, unlike the previous frame holder forceps (21), which was made in a single piece which contained the opening mechanism of the forceps, which needed the only addition of the clip and the cable, in the case of the new sub-frame, this is composed of five elements, the first (22) that contains the micro pulley against which controls the rotation of the frame itself, within which a second member (23) can slide in the direction perpendicular to the axis of the pulley and toward the frame. This second member (23) presents two holes in the direction perpendicular to the sliding axis, within which two shafts of different lengths (24 and 25) held together by a base (26) can move. Since then the longer shaft (24) protrudes from the frame (23), while the element (25) is always internal, by pressing the shaft (24) which protrudes is obtained also the lowering of the second shaft (25) connected to the element (26), which leaves the empty cavity (28), while the sliding of the longer shaft produces no effects regarding coupling to the cable. In this cavity is instead inserted the tab (29) which is connected on the side opposite to the axis (30) of the arms (31) of the quadrilateral opening forceps (32).

A spring (33), also present in the previous frame, as well as quadrilateral and forceps (30-32), holds the forceps open, while the last of the five new elements (34) is the true frame holder forceps, which however is limited to be a C-shaped structure, with four rear protuberances that guide the connection between the latter and the first element (23). How does it work then the coupling and uncoupling of the latter part (34) already fitted inside with all its components (29-33) is quite simple. In fact, just pick up the ring of command of the instrument, to advance the pawl (23) with respect to the bracket (22) while pressing the shaft (24), and at the same time closing the forceps (32) which is protruding the connecting element (29), which is inserted in the cavity (28), in which, by pressing on the base (26) is reinserted, the shaft (25). Note also that the latter has a lower section in the area of engagement, for which it cannot exit if not repeating these operations in reverse. And finally it should be noted that, thanks to the configuration adopted, it is also easy to mount forceps they open both in the plane of opening of the arms that in the plane perpendicular to this, being sufficient to construct a connecting element (35) similar to the previous (29), but that present the two hooking holes parallel between them rather than perpendicular. Of course this is only one way to realize the separation of the forceps with respect to the instrument, and other possible solutions always fall in the more general case of single instrument with two arms equipped with forceps end.

1. Twin surgical instrument for mono access laparoscopy, comprising a main body of bigger diameter and by two arms arranged in parallel and bound by hinges, of a diameter equal to half of the main body, the opening of which is controlled by a system of pulleys or connecting rods characterized in that said system is wholly internal to the main body, for which the surgical instrument, not included in the present patent, can be moved independently from the arms holding forceps, and that in the direction perpendicular the arms may have greater size, provided it is entirely circumscribed by the external dimension of the initial body, bearing at the end two forceps, made integral to secondary arms, these secondary arms leaning inward with movement opposite to the opening of the arms and controlled by a single command, while two secondary controls, allow the independent opening of each of the two forceps, in a way totally independent from the degree of openness of the arms.

2. Twin surgical instrument for mono access laparoscopy as described in claim 1, in which the opening command of the arms is entrusted to a rotating bezel, so as to make this command well differentiated from those of the closure of the forceps, while its actuation is realized by rods pushed ahead by a worm screw moved by the knob.

3. Twin surgical instrument for mono access laparoscopy as described in claim 1, in which the arms holder forceps are moved by connecting rods, pivoted from one part to the fixed part of the body of the instrument and the other on the same holder forceps arms, being the radius of the pivot on which the connecting rod on the fixed part twice the radius of the pin on which rotates on the mobile part, in order to obtain precisely the opposite rotation

4. Twin surgical instrument for mono access laparoscopy as described in claim 1, in which rings are provided with suitable command ratchets adapted to prevent the spontaneous opening of the forceps.

5. Twin surgical instrument for mono access laparoscopy as described in claim 1, in which forceps can be detachable, replaced with tools of a different type, and mounted with the opening in different orientations.
